(19) United States
(12) Patent Application Publication
(10) Pub. No.: US 2010/0092572 A1
(43) Pub. Date: Apr. 15, 2010

(54) CHITOSAN-BASED COLLOIDAL PARTICLES FOR RNA DELIVERY

(76) Inventors: Peter Kaeuper, Lausanne (CH); Carsten Laue, Nyon (CH)

Correspondence Address: Richard Voellmy, Avenue de Sully 67, La Tour-de-Peilz, 1814, CH

(21) Appl. No.: 12/449,057

(22) PCT Filed: Jan. 23, 2008
(86) PCT No.: PCT/IB2008/000170

§ 371 Date: Jul. 22, 2009

Related U.S. Application Data
(60) Provisional application No. 60/898,057, filed on Jan. 29, 2007.

(51) Int. Cl. A61K 9/14; A61K 48/00; C12N 15/00

(52) U.S. Cl. 424/499; 514/44 R; 514/44 A; 435/455; 977/773

(57) ABSTRACT

The present invention provides new colloidal particles of negative zeta potential comprising a ribonucleic acid, a chitosan and a polyanion, and compositions comprising such particles. The compositions are useful for delivery of ribonucleic acids into mammalian cells in vitro, ex vivo and in vivo.

### FIELD OF THE INVENTION

The present invention relates to the fields of polymer chemistry, colloid chemistry, polyelectrolyte chemistry, biomedical engineering and pharmaceutical sciences. More specifically, it concerns a novel polymer-based hydrophilic nanoparticle system for RNA delivery into human or animal cells in vitro and in vivo.

### BACKGROUND OF THE INVENTION

Nano-sized systems are sub-microscopic systems defined by sizes below one micrometer. Systems above one micrometer in size are considered microparticulate. Nanoparticles are used as carrier systems, e.g., for drugs, pro-drugs, proteins, peptides, enzymes, vitamins, etc. For delivery applications, nanoparticles typically are formed in the presence of the molecules to be delivered so that they are encapsulated within the particles for subsequent release.

Hydrophilic nanoparticles can be produced in different ways. One approach is to introduce hydrophilic materials to be delivered inside water droplets of a water-in-oil emulsion. However, this method typically makes use of organic solvents and detergents, i.e., chemicals often not tolerated by complex biological molecules and systems. An attractive approach for producing hydrophilic particles relies on the interactive forces between polyanions and polycations. Particle formation can occur under mild conditions that are not detrimental to complex molecules such as ribonucleic acids. Organic solvents, detergents, and unfavorable acidic or alkaline pH conditions do not need to be utilized. Salts may be present during particle formation.

A favored polycation for pharmaceutical, medical or biotechnological applications is chitosan. Chitosan is a natural polymer composed of glucosamine units. It is produced from crustacean shells or by biotechnological processes. Chitosan is nearly exclusively derived from chitin by a deacetylation process. Both chitin and chitosan are composed of randomly distributed β-(1-4)-linked D-glucosamine (deacetylated unit) and N-acetyl-D-glucosamine (acetylated unit). The two types of polysaccharides differ in the degree of their acetylation and, consequently, in their aqueous solubility under acidic conditions. At degrees of acetylation greater than about 40%, the molecules are insoluble and are referred to as chitin, whereas at lower degrees of acetylation, molecules are soluble and are called chitosan. Chitosan is available from suppliers in a variety of forms. The different forms exhibit different molecular weights and degrees of deacetylation. Furthermore, chitosan is available in the form of different salts. Chitosan is known for its excellent biocompatibility, and is therefore part of many pharmaceutical formulations. Hirano et al. Chitosan: A biocompatible material for oral and intravenous administrations. In: Progress in biomedical polymers. Gebelein and Dunn eds. Plenum Press, New York (1990) pp. 283-289. Chitosan is insoluble in aqueous solutions of neutral pH values, but soluble at slightly acidic pH values. As the molecular weight decreases below about 10,000 g/mol, chitosan becomes more soluble at neutral pH values. Chitosans that are soluble at neutrality are sometimes referred to as oligochitosans. Chae et al. Influence of molecular weight on oral absorption of water-soluble chitosans. Journal of Controlled Release 102 (2005), 383-394. Two recently published review articles underscore the interest in chitosan, particularly in polyelectrolyte complexes of chitosan and a polyanion, for use in biomedical applications. The first of these articles relates to release systems as well as biomedical application of chitosan complexes. Berger et al. Structure and interactions in covalently and ionically crosslinked chitosan hydrogels for biomedical applications. Eur. J. Pharm. Biopharm. 57 (2004), 19-34. The second article provides a detailed account of interactions between chitosan and different polyanions such as anionic polysaccharides, proteins or synthetic polymers with respect to the formed macroscopic structure. Berger et al. Structure and interactions in chitosan hydrogels formed by complexation or aggregation for biomedical applications. Eur. J. Pharm. Biopharm. 57 (2004), 35-52.

Until present, nano-sized vectors based on chitosan and its derivatives intended for ribonucleic acid delivery were designed to exhibit a positive net surface charge. Katas and Alpar. Development and characterization of chitosan nanoparticles for siRNA delivery. Journal of Controlled Release 115 (2006), 216-225; Howard et al. RNA Interference in Vitro and in Vivo Using a Chitosan/siRNA Nanoparticle System. Molecular Therapy 14 (2006), 476-484; Liu et al. The influence of polymeric properties on chitosan/siRNA nanoparticle formulation and gene silencing. Biomaterials 28 (2007), 1280-1288. The net positive surface charge was seen as a prerequisite for successful transfection. To quote from a recent review article “It is widely accepted that the positive charge facilitates binding to cell membrane, which is not surprising since cell membrane is negatively charged.” Liu and Yao. Chitosan and its derivatives—a promising non-viral vector for gene transfection. J. Contr. Release, 83 (2002), 1-11. Furthermore, ribonucleic acids are rapidly degraded by ribonucleases, in particular when administered in vivo. It could be reasoned that ribonucleic acids may be capable of being protected by inclusion in nanoparticles in which they are stabilized by electrostatic interactions in the presence of an excess of a polycation.

However, chitosan-based particles with positive surface charge or zeta potential are unstable in media containing salts. Furthermore, the presence of serum proteins also leads to instability. Kaeuper and Forrest. Chitosan-based nanoparticles by ionotropic gelation. XIV International Workshop on Bioencapsulation. Wandrey and Poncelet eds. (2006), pp. 69-72. Accordingly, there is a need for chitosan-based nanoparticles comprising ribonucleic acids that exhibit an acceptable degree of stability in saline environments as well as in the presence of serum proteins, and that are capable of delivering the ribonucleic acids into the cytoplasm of cells and effect this delivery in such a way that the ribonucleic acids retain their intended biological activity inside the cells.

### SUMMARY OF THE INVENTION

The present invention relates to colloidal particles, each particle comprising a chitosan, a ribonucleic acid and a polyanion, whereby the positively charged component, chitosan, and the negatively charged components, ribonucleic acid and anion, are present in proportions or are distributed in the particles in a fashion that results in a negative zeta potential. A negative zeta potential is determined by electrophoretic mobility measurements and represents a net negative surface charge of the particle. Preferred sizes for the colloidal particles are between about 10 nanometer and one micrometer. Chitosan types with a wide range of molecular weights from about 1,000 to 1,000,000 g/mol can be utilized in the particles of the invention. Preferred is a chitosan with a molecular weight from about 10,000 to 100,000 g/mol, or from about 1,000 to 10,000 g/mol. At acidic pH values, chitosan exhibits a polycationic character. Polyanions comprised in the colloidal particles are molecules that exhibit a plurality of negative charges at pH values above pH 6. Preferred polyanions are adenosine triphosphate, tripolyphosphate, alginate, PEGylated alginate, hyaluronate, PEGylated hyaluronate, chondroitin sulfate, carboxymethyl cellulose, and dextran sulfate. Most preferred are adenosine triphosphate, alginate and chondroitin sulfate. The particles of the invention may also contain a plurality of different polyanion, preferably selected from the group consisting of adenosine triphosphate, tripolyphosphate, alginate, PEGylated alginate, hyaluronate, PEGylated hyaluronate, chondroitin sulfate, carboxymethyl cellulose, and dextran sulfate. Most preferred are the combinations of chondroitin sulfate and alginate, and of adenosine triphosphate and alginate. The ribonucleic acid contained in the particles may be any ribonucleic acid. Preferred ribonucleic acids are those that can exert a biological function or effect, including messenger RNAs, self-replicating messenger RNAs, interfering RNAs and antisense RNAs. Particles of the invention can further comprise one or more substances selected from the group consisting of a multivalent cation, an uncharged polymer, an uncharged saccharide and a biologically active substance other than a ribonucleic acid.

Other embodiments of the invention concern compositions for ribonucleic acid transduction that comprise any kind of particle of the invention, including those that were characterized before as preferred, comprising one or more of a chitosan of the preferred molecular mass ranges of about 10,000 to 100,000 g/mol and about 1,000 to 10,000 g/mol, a polyanion or a plurality of polyanions, preferably selected from the group of adenosine triphosphate, tripolyphosphate, alginate, PEGylated alginate, hyaluronate, PEGylated hyaluronate, chondroitin sulfate, carboxymethyl cellulose, and dextran sulfate, and more preferably selected from chondroitin sulfate, adenosine triphosphate, or chondroitin sulfate or adenosine triphosphate and alginate, and a ribonucleic acid, preferably selected from messenger RNAs, self-replicating messenger RNAs, interfering RNAs and antisense RNAs. The compositions may also include an excipient. Excipients can include a salt, an isotonic agent, a serum protein, a buffer or other pH-controlling agent, an anti-oxidant, a thickener, an uncharged polymer, a preservative or a cryoprotectant. The compositions can also include a biologically active substance other than a ribonucleic acid such as a drug, a pro-drug, or a therapeutic or otherwise biologically active peptide or protein.

Further embodiments relate to uses of the compositions of the invention for transducing mammalian cells with a ribonucleic acid. These uses comprise contacting a cell to be transduced with a composition of the invention that comprises particles of the invention, which particles contain the ribonucleic acid to be transduced. In the context of the present invention, the term “transduction” refers to the process of delivering a particle or an RNA molecule into a cell. Administration of a composition of the invention to cultured cells (in vitro), cells retrieved from a mammalian organism (ex vivo) or cells residing in a mammalian organism (in vivo) causes delivery of the ribonucleic acid contained in the composition into the cultured cells, the cells retrieved from the organism or the cells residing in the organism, as the case may be. Specific embodiments include a method for expressing a protein of interest in a mammalian cell, comprising contacting the cell with a composition of the invention that includes a messenger RNA or a self-replicating messenger RNA encoding the protein of interest, a method for inhibiting expression of a gene of interest in a mammalian cell, comprising contacting the cell with a composition of the invention comprising an interfering RNA directed to a transcript of the gene of interest, as well as a method for inhibiting expression of a gene of interest in a mammalian cell, comprising contacting the cell with a composition of the invention comprising an antisense RNA that is complementary to a transcript of the gene of interest.

Another set of embodiments relates to processes for producing the colloidal particles of the invention. In one such process, a first aqueous solution of a chitosan and a second aqueous solution of a ribonucleic acid and a polyanion (or a plurality of anions) are prepared, and the first solution is added slowly to the second solution such that, after addition, the number of negative charges on the resulting particles exceeds that of positive charges, i.e., particles of negative zeta potential are formed. In an alternative process, a first aqueous solution of a ribonucleic acid and, optionally, a first polyanion (or polyanions) and a second aqueous solution of a chitosan are prepared. The first solution is slowly added to the second solution, causing formation of a dispersion, from which uncomplexed chitosan may be removed. In a further step, to an aqueous solution of a second polyanion (or polyanions) is added the dispersion such that, after addition, the number of negative charges on the particle surface exceeds that of positive charges. In yet another process, aqueous solutions of chitosan and a first polyanion (or polyanions) are combined to form a first dispersion, from which uncomplexed chitosan may be removed. To this first dispersion a solution of a ribonucleic acid and, optionally, a second polyanion is added, causing formation of a second dispersion. To a third aqueous solution of a polyanion (or polyanions) is then added the second dispersion, such that, after addition, the number of negative surface charges on the particles exceeds that of positive charges. The first, second or third polyanions in the above processes may be identical or different.

### DETAILED DESCRIPTION OF THE INVENTION

The present invention relates to colloidal particles comprising a chitosan, a ribonucleic acid and a polyanion, whereby the positively charged component, chitosan, and the negatively charged components, ribonucleic acid and anion, are present in relative amounts or are distributed in such a way that particles of negative zeta potential are formed. These particles represent new vehicles for effectively introducing ribonucleic acids into cells. A negative zeta potential is determined by electrophoretic mobility measurements and represents a net negative surface charge of the particle.

The colloidal particles of the present invention offer several advantages over other types of nanoparticles described in the prior art, e.g., covalently cross-linked chitosan nanoparticles prepared by oil-in-water emulsion techniques or a liposomal approach. Their preparation is simple and does not require any potentially harmful ingredients and solvents such as organic solvents, oils and aldehydic cross-linking agents for incorporating a ribonucleic acid in the nanoparticle. Partners of different charges have to react in order to obtain the colloidal particles of the invention by polyelectrolyte complex formation. Of primary importance is the choice of the cationic partner. As positive charges tend to react with many anionic surfaces or negatively charged biomolecules present in biological environments, the presence of cationic charges is a potential source of toxicity of such material. The positive charges on chitosan persist only at pH levels below physiological values. This is one reason why chitosan exhibits good biocompatibility. Together with a ribonucleic acid and an appropriate polyanion, chitosan forms highly biocompatible and potentially degradable colloidal particle systems. It is a key characteristic of the colloidal particles of the invention that they have negative zeta potential. Negative zeta potential improves stability of the particles in physiological environments in which negatively charged surfaces such as cell membranes and serum proteins abound. Surprisingly, the negative zeta potential neither prevents delivery of ribonucleic acids into cells by the particles of the invention nor does it negatively affect the ability of the transported ribonucleic acids to exert their intended biological functions or effects.

Depending on conditions under which they are produced, colloidal particles in the nanometer to micrometer ranges can be obtained. Preferred colloidal particles of the invention are nanoparticles having an average diameter of between about 10 and 1000 nm.

Different types of chitosan can be used in a particle of the invention. The chitosans may differ in average molecular weight, distribution of molecular weights, degree of deacetylation, acetylation pattern, type of anionic counterion and purity. Regarding molecular size, chitosans with molecular weights from 1,000 to 1,000,000 g/mol can be used in the particles of the invention. The lower end of this range (below molecular weights of approximately 10,000 g/mol) includes molecules that are also referred to as oligochitosans and are characterized by solubility in aqueous solutions at pH values higher than 6. Preferred molecular weights of the chitosans used in the particles of the invention are from 1,000 to 10,000 g/mol and from 10,000 to 100,000 g/mol Typically, chitosans will be present in amounts exceeding 10% of the weight of the particles. Chitosans are produced from crustacean shells or by biotechnological processes. Commercial sources of chitosans are, e.g., Primex Ltd. (Iceland), Marinard Ltd. (Canada) or FMC Biopolymers (U.S.) as producers of crustacean-based chitosans, and Kitozyme Ltd. (Belgium) as producer for biotechnologically derived chitosan. Chitosans used in the particles of the invention can also be chemically modified on their hydroxyl or on their amino functionality. Such derivatized chitosans can be used instead or in combination with unmodified chitosans. Examples of moieties linked to the chitosan molecule are fluorescence markers such as fluorescein, anionic groups such as carboxymethyl, neutral synthetic small molecular weight chains such as polyethylene glycol (PEG) chains and saccharides such as mono- or oligo-saccharides such as mannose and galactose. Modifications on the chitosan's amino functions can be executed in order to obtain secondary, tertiary or quaternary amines. The latter is of particular interest as a pH independent positive charge can be integrated in the chitosan molecule. Prominent derivatives are the trialkyl chitosans, such as trimethyl chitosan. Of course, a person skilled in the art may choose other polycations, which can be used together with or instead of a chitosan. Examples are polyethylene imine, polyethylene imine derivatives, poly(methylene-co-guanidine) and poly-L-lysine.

The ribonucleic acid comprised in a particle of the invention can be a ribonucleic acid of any chain length greater than about four nucleotides. The term “ribonucleic acid” is meant to include ribonucleic acids as well as derivatives and different salts. A ribonucleic acid can be a single species with a distinct base sequence, two species with base sequence complementarity or a mixture of two or more kinds of molecules with different, non-complementary base sequences. They can be isolated from cells, made by synthetic methods known in the art or transcribed in vitro. RNAs that can be used in particles of the invention are double stranded RNA (dsRNA), single stranded RNA (ssRNA). Preferred ribonucleic acids are RNAs that perform a biological function when introduced into cells such as messenger RNAs and self-replicating mRNAs, also referred to as replicon RNA. Also preferred are ribonucleic acids that have biological effects when introduced into cells such as antisense RNAs or interfering RNA, including long double-stranded RNA and small interfering RNA (siRNA), that can inhibit the function of an RNA endogenous to a cell containing a sequence that can hybridize or otherwise form a complex with the interfering RNA or antisense RNA.

The polyanion comprised in a particle of the invention can be any anion containing a plurality of negative charges at the pH value at which particle formation occurs. Specific examples of useful polyanions include the sulfate anion, oligophosphates such as tripolyphosphate (TPP), nucleoside triphosphate including adenosine triphosphate (ATP), nucleoside diphosphates including adenosine diphosphate (ADP), poly-acrylic acid, chondroitin sulfate, alginate, hyaluronate, dextran sulfate, heparin, heparan sulfate, gellan gum, pectin, kappa, lamda and iota carrageenan, xanthan and derivatives thereof; sulfated, carboxymethylated, carboxyethylated or sulfoethylated varieties of glucans or xylans, glucan or xylan derivatives, glucosaminoglucans or glucosaminoglucan derivatives; proteins like collagen and keratose. All of these example polyanions are available from various commercial suppliers or can be synthesized by those skilled in the art using known methodology. Preferred polyanions are adenosine triphosphate, tripolyphosphate, alginate, hyaluronate, chondroitin sulfate, carboxymethyl cellulose and dextran sulfate. Most preferred are chondroitin sulfate, adenosine triphophate and alginate.

Polyanions used in particles of this invention can also be modified to carry targeting ligands. A targeting ligand is a moiety that binds to specific surface features of cells. Examples of targeting ligands are saccharides, liposaccharides, antibodies, cell adhesion molecules, hormones and neurotransmitters. Furthermore, polyanions can be modified by moieties that do not specifically interact with cells. Such non-interacting moieties can be polyethylene glycol units of different molar mass with different termini. Examples of such termini are hydroxy and methoxy groups. Moreover, polyanions of this invention can be modified to carry targeting ligands linked to the polyanion via a spacer such as polyethylene glycol. Such modifications may be made using the carbodiimde reaction for linking carboxyl and amine functionalities to form amide bonds. For example, a carboxyl group of the polyanion can be reacted with a terminal amine of a polyethylene glycol molecule; a bifunctional polyethylene glycol molecule can be reacted with both a targeting ligand and a polyanion. These reaction pathways are known under the term PEGylation.

Colloid particles of the invention can be obtained readily by drop-wise addition of an aqueous solution comprising one component of the particles to an aqueous solution containing another component of opposite charge and gentle agitation.

No particular attention needs to be paid to the size of the droplets or the flow rate of addition of the first solution to the second solution. Formation of the particles of the present invention occurs spontaneously by colloid formation of the system's anionic components and chitosan. Particle formation results in the so-called “Tyndall effect” that can be detected by the human eye. The solvent system for the component solutions can be water or salt solutions. Conditions of pH can be varied depending on the type of chitosan used and can include physiological pH values. Chitosans of molar weights above approx. 10,000 g/mol require slightly acidic pH values, preferably between pH 4.5-6.6, whereas chitosan of molar weights below 10,000 g/mol have a wider pH range in complex formation, pH 4.5-7.5. Within certain limits, water-miscible solvents can be present, e.g., alcohols such as methanol, ethanol, 2-propanol, or N-butanol, can be present at concentrations of up to about 20% (v/v). This process of particle formation can also be considered as ionic gelation, ionic cross-linking, co-acervation or polyelectrolyte complex formation. Chitosan polyelectrolyte complex formation has been extensively described in Berger et al. Structure and interactions in chitosan hydrogels formed by complexation or aggregation for biomedical applications. J. Pharm. Biopharm. 57 (2004), 35-52 and Agnihotri et al. Recent advances on chitosan-based micro- and nanoparticles in drug delivery. Journal of Controlled release 100 (2004), 5-28.

The number and order of steps that are performed to produce particles of the invention can be varied. For example, a solution containing one or more polyanions and a ribonucleic acid may be combined as described above with a solution of a chitosan. Amounts of components combined are chosen such that particles with negative zeta potential result from polyelectrolyte complex formation. Another method is to combine a solution comprising a ribonucleic acid and, optionally, a polyanion with a solution comprising a chitosan such that colloidal particles of positive zeta potential are obtained. If necessary, an excess of uncomplexed chitosan can be removed by processes such as dialysis, ultrafiltration and centrifugation. Thereafter, the dispersion of particles of positive zeta potential is combined with a solution comprising one or more polyanions, forcing conversion of the particles with positive zeta potential to particles with negative zeta potential. It is noted that the two or more polyanions that are incorporated in the final particles may be the same or may be different. A variation of the previous method is to produce a first dispersion of colloidal particles with positive zeta potential by combining a solution of chitosan and a solution of one or more polyanions. After removal of excess chitosan, should there be an excess, the first dispersion is combined with a solution comprising a ribonucleic acid and, if desired, one or more polyanions to produce a second dispersion, still of positive zeta potential. This second dispersion is then added to a solution of one or more polyanions to force conversion to particles with negative zeta potential. It is noted that additional components can be added during particle formation. Examples of such additional components are multivalent cations such as calcium, uncharged polymers such as polyethylene glycol, or uncharged saccharide derivatives. Additional components may also include one or more biologically active substances. Such biologically active substances may be any biologically active substance, including small-molecule drugs or pro-drugs and therapeutic or otherwise biologically active peptides or proteins, provided that they are soluble in aqueous solutions at concentrations exceeding the concentrations at which they are therapeutically active or exert their other biological activity. Specific examples of such biologically active substances are NSAIDs, preferably NSAIDs belonging to the classes of salicylates, aryl alkanoic acids, 2-aryl propionic acids, N-aryl anthranilic acids, pyrazolidine derivatives, oxicams, coxibs and sulphonanilides.

The size of the colloidal particles of the invention can range from the low nanometer range to the low micrometer range. Particle size is influenced by the nature of the polyanion or polyanions employed, the concentations of anionic component or components and ribonucleic acid in the complexation reaction, the presence and concentration of salts, the presence, nature and concentration of added uncharged polymers (Calvo et al. Novel Hydrophilic Chitosan-Polyethylene Oxide Nanoparticles as Protein Carriers. Journal of Applied Polymer Science, 63 (1997), 125-132), the molar mass and degree of acetylation of chitosan (Douglas et al. Effect of experimental parameters on the formation of alginate-chitosan nanoparticles and evaluation of their potential application as DNA carrier. Journal of Biomaterials Science, 16 (2005), 43-56; Liu et al. The influence of polymeric properties on chitosan/siRNA nanoparticle formulation and gene silencing. Biomaterials, 28 (2007), 1280-1288) and temperatures of different complexation steps. Typically, particles formed by the processes described above are of somewhat heterogeneous size. It is possible to obtain populations of particles with more homogeneous sizes by selection subsequent to preparation by means of filtration, ultrafiltration, dialysis or centrifugation, or combinations of these methods.

Solutions containing colloid particles of the invention can be subjected to solvent changes, purification (e.g., dialysis), wet heat sterilization, and desiccation (e.g., freeze drying and spray drying).

The present invention also relates to compositions for transduction of functionally intact ribonucleic acids into isolated cells, either grown in culture (in vitro) or obtained from a mammalian organism (ex vivo), or into cells of a mammalian organism in vivo. Such compositions comprise colloidal particles of the invention containing the ribonucleic acid to be transduced in an aqueous solution that may, optionally, contain one or more excipients. While such excipients may be present in compositions that are used for transduction of cells in vitro, they are predictably of greater importance in compositions that are administered to mammalian animals or a human patient in vivo. The excipient can be a physiologically acceptable salt. A physiologically acceptable salt is any salt that does not diminish the biological activity or effect of the composition of the invention and does not impart any deleterious or ontoward effects on the animal or human patient to which it is administered as part of the composition.

Excipients used in compositions of the invention may further include an isotonic agent and a buffer or other pH-controlling agent. These excipients may be added for the attainment of preferred ranges of pH (about 6.0-8.0) and osmolarity (about 50-300 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the invention may further contain a preservative. Examples preservatives are polyhexamethylene-biguanidine, benzalkonium chloride, stabilized oxychloro complexes (such as those known as PuriteR), phenylmercuric acetate, chlorobutanol, sorbic acid, chlorhexidine, benzyl alcohol, parabens, and thimerosal. Typically, such preservatives are present at concentrations from about 0.001 to 1.0%.

Furthermore, the compositions of the invention may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 5%.

The compositions of the invention may also contain a viscosity-increasing or thickening agent. Preferred thickening agents are cellulose and cellulose-derivative thickening agents such as alkyl celluloses and hydroxyalkyl celluloses. Examples for this type of thickening agent are methyl cellulose and hydroxypropyl methylcellulose (e.g., Nos. 2208 or 2906 as defined in the Japanese and U.S. Pharmacopeia). Other thickening agents include polyvinyl polymers and polyvinylpyrrolidones. Example polyvinyl polymers are polyvinylacetates and polyvinylalcohols, and example polyvinylpyrrolidones are poly-N-vinylpyrrolidones and vinylpyrrolidone co-polymers. The compositions of the invention may further comprise an anti-oxidant. Anti-oxidants that may be acceptable include sodium metabisulfite, sodium thiosulfate, acetylcysteine, butylated hydroxyanisole, and butylated hydroxytoluene. Typically, the concentration of an anti-oxidant is within the range from about 0.0001 to about 0.01% (w/v). Moreover, the compositions may contain serum proteins for stabilization. An example protein that can be utilized for this purpose is serum albumin.

Finally, additional components of compositions of the invention can be uncharged polymers such as polyethylene glycol, uncharged saccharide derivatives, or one or more biologically active substances. Such biologically active substance may be any biologically active substance, including small-molecule drugs or pro-drugs and therapeutic or otherwise biologically active peptides or proteins. Specific examples of such biologically active substances are NSAIDs, preferably NSAIDs belonging to the classes of salicylates, aryl alkanoic acids, 2-aryl propionic acids, N-aryl anthranilic acids, pyrazolidine derivatives, oxicams, coxibs and sulphonanilides.

The present invention also relates to methods of transduction of mammalian cells in vitro, ex vivo and in vivo with a functionally intact ribonucleic acid. These methods involve contacting the cells to be transduced with a composition of the present invention that comprises colloidal particles containing the ribonucleic acid to be transduced. RNAs foreseen for in vitro transduction are applied in concentrations from about 1 pmol to 1 mmol RNA per 2×10⁶ cells, and preferably in concentrations from about 10 pmol to 10 nmol RNA per 2×10⁶ cells. For in vivo transduction, the RNA concentration can be from 5 pmol to 5 mmol RNA per kg body weight, and preferably from about 50 pmol to 50 nmol RNA per kg body weight. The proportion of RNA per nanoparticle is limited by the number of potential positive charges of the chitosan molecules, which depends on the degree of deacetylation of the chitosan utilized and the pH during complexation with the RNA. The number of negative charges of the RNA molecules is preferably below 80% of the number of positive charges provided by the chitosan, and most preferably from about 1% to 30%.

The invention is further elaborated by the following examples. The examples are provided for purposes of illustration to a person skilled in the art and are not intended to be limiting the scope of the invention as described in the claims. Thus, the invention should not be construed as being limited to the examples provided, but should be construed to encompass any and all variations that become evident as a result of the teaching provided herein.

### EXAMPLES

### Particle Forming Materials

Chondroitin sulfate type A, TPP and ATP were purchased at Sigma-Aldrich (Sigma-Aldrich, Germany) and used without further purification. Hyaluronate of molecular weight of approx. 170 kg/mol was purchased at Lifecore (Lifecore, U.S.). Alginate of low and middle viscosity was of an in-house purified quality. Chitosan of approx. 50 kg/mol and of approx. 100 kg/mol was purchased at Sigma-Aldrich (Sigma-Aldrich, Germany) and subjected to purification prior to use. Reduced molecular weight chitosan, i.e. molecular weight of approx. 5 kg/mol, was in-house produced.

### Example 1

### Preparation of Colloidal Particles with Negative Zeta Potential Containing Chitosan, mRNA and Chondroitin Sulfate

Preparation of Rhodamine-Labeled Enhanced Green Fluorescence Protein (EGFP) Expressing mRNA:

A plasmid containing a cDNA for enhanced green fluorescence protein functionally linked to a bacteriophage T7 promoter (pSLTM3B-EGFP) was linearized by restriction digestion with Aat II (New England Biolabs, U.S.), and purified by Qiagen gel extraction kit (Qiagen, Switzerland). Transcription was performed using the Megascript kit (Ambion, UK) to generate RNA from the linearized plasmid. Transcripts were labelled with rhodamine using the Label-It reagent (Mirus, U.S.) following the manufacturer's instructions (50 μL of RNA at a concentration of 0.1 μg/μL incubated with 50 μL of labelling reagent for 1 h at 37° C. and purified by ethanol precipitation).

At room temperature, a solution of 70 μL of 0.025% chitosan (molecular weight approx. 50 kg/mol, subjected to purification prior to use) in aqueous HCl at pH 4.6 was added drop-wise under gentle agitation to a solution of 420 μL of 0.1% chondroitin sulfate and 10 μg of rhodamine-labeled EGFP expressing mRNA in water at pH 7.0. After 1 h of gentle agitation, the resulting dispersion was filtered through a 1.2 μm filter (mixed cellulose ester membrane (Sartorius, Germany) and then dialyzed against water using a 100,000 g/mol MWCO dialysis membrane (Spectrum Laboratories, U.S.). The zeta potential was measured at less than −10 mV.

### Example 2

### Preparation of Colloidal Particles with Negative Zeta Potential Containing Chitosan, mRNA and Adenosine Triphosphate and Hyaluronic Acid Sodium Salt

Preparation of Rhodamine-Labeled EGFP Expressing mRNA:

A plasmid containing a cDNA for enhanced green fluorescence protein functionally linked to a bacteriophage T7 promoter (pSLTM3B-EGFP) was linearized by restriction digestion with Aat II (New England Biolabs, U.S.), and purified by Qiagen gel extraction kit (Qiagen, Switzerland). Transcription was performed using the Megascript kit (Ambion, UK) to generate RNA from the linearized plasmid. Transcripts were labelled with rhodamine using the Label-It reagent (Mirus, U.S.) following the manufacturer's instructions (50 μL of RNA at a concentration of 0.1 μg/μL incubated with 50 μL of labelling reagent for 1 h at 37° C. and purified by ethanol precipitation).

10 μg rhodamine-labeled EGFP expressing mRNA was dissolved in 100 μL of 0.1% adenosine triphosphate in water at pH7. At room temperature, this solution was added drop-wise under gentle agitation to a solution of 2 mL of 0.025% chitosan (molecular weight approx. 100 kg/mol, subjected to purification prior to use) in aqueous HCl at pH 5.5. Opalescence appeared after the first added drops and became increasingly intense. After 1 h of gentle agitation, the dispersion was filtered through a 1.2 μm filter (mixed cellulose ester membrane, Sartorius, Germany) and dialyzed against water using a 0.05 μm hollow fiber module (KrosFlo module, polysulfone membrane, Spectrum Laboratories, U.S.). A milky, opalescent dispersion with visible Tyndall effect resulted, which remained unchanged after filtration through 1.2 μm and 0.8 μm filters (mixed cellulose ester membrane, Sartorius, Germany). Zeta potential was higher than +10 mV. The dispersion containing particles of positive zeta potential was added drop-wise to a solution of 7 mL of 0.05% hyaluronic acid sodium salt in water at pH 7. After 1 h of gentle agitation, the dispersion was dialyzed against water using a 400 kD hollow fiber module (KrosFlo module, polysulfone membrane, Spectrum Laboratories, U.S.)and concentrated to 1 mL. A milky, opalescent dispersion with visible Tyndall resulted, which remained unchanged after filtration through a 1.2 μm filter (mixed cellulose ester membrane, Sartorius, Germany). The zeta potential was measured at less than −10 mV.

### Example 3

### Preparation of Colloidal Particles with Negative Zeta Potential Containing Oligochitosan, mRNA and Adenosine Triphosphate and Sodium Alginate

Preparation of Rhodamine-Labeled EGFP Expressing mRNA:

A plasmid containing a cDNA for enhanced green fluorescence protein functionally linked to a bacteriophage T7 promoter (pSLTM3B-EGFP) was linearized by restriction digestion with Aat II (New England Biolabs, U.S.), and purified by Qiagen gel extraction kit (Qiagen, Switzerland). Transcription was performed using the Megascript kit (Ambion, UK) to generate RNA from the linearized plasmid. Transcripts were labelled with rhodamine using the Label-It reagent (Mirus, U.S.) following the manufacturer's instructions (50 μL of RNA at a concentration of 0.1 μg/μL incubated with 50 μL of labelling reagent for 1 h at 37° C. and purified by ethanol precipitation).

At room temperature, a solution of 100 mL of 0.1% adenosine triphosphate in water at pH 7.0 was added drop-wise under mechanical agitation to a solution of 2000 mL of 0.025% oligochitosan (Mₙ, 4500 g/mol, M_{w} 6000 g/mol) in aqueous HCl at pH 5.5. Opalescence appeared after the first added drops and became increasingly more intense. After 1 h of gentle agitation, the dispersion was filtered through a 1.2 μm filter (mixed cellulose ester membrane, Sartorius, Germany), crossflow-dialyzed against water using a 0.05 μm hollow fiber module (KrosFlo module, polysulfone membrane, Spectrum Laboratories, U.S.) and concentrated to 300 mL. At room temperature, to 3 mL of this dispersion was slowly added under gentle stirring a solution of 10 μg rhodamine-labeled EGFP expressing mRNA in 10 μL water at pH 7, followed by 1 h of gentle agitation. The resulting milky, opalescent dispersion had visible Tyndall effect, which remained unchanged after filtration through 1.2 μm and 0.8 μm filters (mixed cellulose ester membrane, Sartorius, Germany). Zeta potential was found to be greater than +10 mV. Subsequently, at room temperature, the dispersion was added to 5 mL of 0.05% sodium alginate (low viscosity type) in water at pH 7, followed by 1 h of gentle agitation. The dispersion was crossflow-dialyzed against water using a 400 kD hollow fiber module (KrosFlo module, polysulfone membrane, Spectrum Laboratories, U.S.) and concentrated to 1 mL. The resulting milky, opalescent dispersion had visible Tyndall effect that resisted filtration through 1.2 μm and 0.8 μm filters (mixed cellulose ester membrane, Sartorius, Germany). Zeta potential was less than −10 mV.

### Example 4

### Transduction and Demonstration of Translatability of Rhodamine-Labeled Green Fluorescent Protein (GFP)-Expressing mRNA

Preparation of Rhodamine-Labeled EGFP Expressing mRNA:

A plasmid containing a cDNA for enhanced green fluorescence protein functionally linked to a bacteriophage T7 promoter (pSLTM3B-EGFP) was linearized by restriction digestion with Aat II (New England Biolabs, U.S.), and purified by Qiagen gel extraction kit (Qiagen, Switzerland). Transcription was performed using the Megascript kit (Ambion, UK) to generate RNA from the linearized plasmid. Transcripts were labelled with rhodamine using the Label-It reagent (Mirus, U.S.) following the manufacturer's instructions (50 μL of RNA at a concentration of 0.1 μg/μL incubated with 50 μL of labelling reagent for 1 h at 37° C. and purified by ethanol precipitation).

### Preparation of RNA-Nanoparticles:

At room temperature, a solution of 100 mL of 0.1% tripolyphosphate in water at pH 7.0 was added drop-wise under mechanical agitation to a solution of 2000 mL of 0.025% chitosan (middle viscosity) in aqueous HCl at pH 5.5. Opalescence appeared after the first added drops and became increasingly more intense. After 1 h of gentle agitation, the dispersion was filtered through a 1.2 μm filter (mixed cellulose ester membrane, Sartorius, Germany), crossflow-dialyzed against water using a 0.05 μm hollow fiber module (KrosFlo module, polysulfone membrane, Spectrum Laboratories, U.S.) and concentrated to 300 mL. At room temperature, to 12 μL of this dispersion was slowly added under gentle stirring a solution of 5 μg rhodamine-labeled EGFP expressing mRNA in 10 μL water at pH 5, followed by 1 h of gentle agitation. The final volume was adjusted with water at pH 5 to 50 μL. Zeta potential was found to be greater than +10 mV. Subsequently, at room temperature, the dispersion was added to 100 μL of 0.05% alginate (low viscosity type) in water at pH 7, followed by 1 h of gentle agitation. The dispersion was crossflow-dialyzed against water using a 400 kD hollow fiber module (KrosFlo module, polysulfone membrane, Spectrum Laboratories, U.S.). Zeta potential was less than −10 mV.

### Preparation of Monocyte-Derived Dendritic Cells:

Porcine monocyte dendritic cells (MoDCs) were derived from immature precursors obtained from bone marrow aspirate of pigs. Subsequent to depletion of erythrocytes and granulocytes by centrifugation over Ficoll-Paque (1,077 g/L) at 1000 g for 40 min at room temperature, monocytes were isolated by adherence to plastic for 16 h. Monocytes were cultured in phenol red-free Dulbecco's modified Eagle's medium (DMEM) supplemented with 2 mM glutamine, 100 U/mL penicillin, 100 μg/mL streptomycin, 50 μM 2-mercaptoethanol and 10% (v/v) fetal calf serum (FCS). For the generation of MoDC, the medium was further supplemented with 150 ng/mL recombinant plasmid granulocyte-macrophage colony stimulating factor (GM-CSF), 100 U/mL recombinant plasmid interleukin-4 (IL-4) and porcine serum (MoDC medium).

MoDC were generated by culture of monocytes (0.5×10⁶ cells/mL) in the latter MoDC medium for 6 days. On days 2 and 4, half of the MoDC medium was replaced by fresh MoDC medium.

### In Vitro Transduction:

To 150 μL of the nanoparticle dispersion was added, 50 μL of phosphate buffer and sodium chloride solution to make the final solution 5 mM phosphate buffered at pH 7.4 and to have a final sodium chloride concentration of 0.9%. Prior to the in vitro experiments, the dispersion was heated to 37° C. At 37° C., the dispersion (200 μl) was diluted with medium (complete DMEM) to result in 800 μl final volume of which 200 μl were incubated for 48 h with 2×10⁵ monocyte-derived dendritic cells. At the end of this incubation period, supernatant was removed and the cells were washed and analyzed by confocal fluorescence microscopy. Red fluorescence was observed in over 90% of cells, indicating that the rhodamine-labeled EGFP-expressing mRNA was delivered into almost all cells. More important, over 65% of cells exhibited green fluorescence, indicating that in a majority of cells EGFP was expressed at levels sufficiently elevated for fluorescence detection.

1. Colloidal particle having a negative zeta potential comprising a chitosan, a polyanion and a ribonucleic acid, whereby the chitosan, the polyanion and the ribonucleic acid are present in proportions or are distributed in the particle such that the particle has a negative zeta potential.

2. The colloidal particle of claim 1, wherein the particle has a diameter between about 10 and 1000 nanometers.

3. The colloidal particle of claim 1, wherein the chitosan has a molecular weight from about 1000-1,000,000 g/mol.

4. The colloidal particle of claim 1, wherein the chitosan has a molecular weight from about 10,000-100,000 g/mol.

5. The colloidal particle of claim 1, wherein the chitosan has a molecular weight from about 1000-10,000 g/mol.

6. The colloidal particle of claim 1, wherein the polyanion is selected from the group consisting of adenosine triphosphate, tripolyphosphate, alginate, PEGylated alginate, hyaluronate, PEGylated hyaluronate, chondroitin sulfate, carboxymethyl cellulose, and dextran sulfate.

7. The colloidal particle of claim 1, wherein the polyanion is chondroitin sulfate.

8. The colloidal particle of claim 1, wherein the polyanion is adenosine triphosphate.

9. The colloidal particle of claim 1 comprising a plurality of different anions.

10. The colloidal particle of claim 9, wherein each of the polyanions is selected from the group consisting of adenosine triphosphate, tripolyphosphate, alginate, PEGylated alginate, hyaluronate, PEGylated hyaluronate, chondroitin sulfate, carboxymethyl cellulose, and dextran sulfate.

11. The colloidal particle of claim 9 wherein the anions are chondroitin sulfate and alginate.

12. The colloidal particle of claim 9 wherein the anions are ATP and alginate.

13. The colloidal particle of claim 1, wherein the ribonucleic acid is selected from the group consisting of a messenger RNA, a self-replicating messenger RNA, an interfering RNA, and an antisense RNA.

14. The colloidal particle of claim 1, wherein the ribonucleic acid is a messenger RNA.

15. The colloidal particle of claim 1, wherein the ribonucleic acid is a self-replicating messenger RNA.

16. The colloidal particle of claim 1, wherein the ribonucleic acid is an interfering RNA.

17. The colloidal particle of claim 1, wherein the ribonucleic acid is an antisense RNA.

18. The colloidal particle of claim 1 that further comprises one or more substances selected from the group consisting of a multivalent cation, an uncharged polymer, an uncharged saccharide and a biologically active substance other than a ribonucleic acid.

19. A composition for ribonucleic acid transduction comprising in an aqueous solution a colloidal particle having a negative zeta potential comprising a chitosan, a polyanion, a ribonucleic acid and, optionally, an excipient, whereby the chitosan, the polyanion and the ribonucleic acid are present in proportions or are distributed in the particle such that the particle has a negative zeta potential.

20. The composition of claim 19, wherein the diameter of the colloidal particle is between about 10 and 1000 nanometers.

21. The composition of claim 19, wherein the chitosan comprised in the colloidal particle has a molecular weight from about 1,000-1,000,000 g/mol.

22. The composition of claim 19, wherein the chitosan comprised in the colloidal particle has a molecular weight from about 10,000-100,000 g/mol.

23. The composition of claim 19, wherein the chitosan comprised in the colloidal particle has a molecular weight from about 1,000-10,000 g/mol.

24. The composition of claim 19, wherein the polyanion comprised in the colloidal particle is selected from the group consisting of adenosine triphosphate, tripolyphosphate, alginate, PEGylated alginate, hyaluronate, PEGylated hyaluronate, chondroitin sulfate, carboxymethyl cellulose, and dextran sulfate.

25. The composition of claim 19, wherein the polyanion comprised in the colloidal particle is chondroitin sulfate.

26. The composition of claim 19, wherein the polyanion comprised in the colloidal particle is adenosine triphosphate.

27. The composition of claim 19, wherein the colloidal particle comprises a plurality of different anions.

28. The composition of claim 27, wherein each of the polyanions comprised in the colloidal particle is selected from the group consisting of adenosine triphosphate, tripolyphosphate, alginate, PEGylated alginate, hyaluronate, PEGylated hyaluronate, chondroitin sulfate, carboxymethyl cellulose, and dextran sulfate.

29. The composition of claim 27, wherein the anions comprised in the colloidal particle are chondroitin sulfate and alginate.

30. The composition of claim 27, wherein the anions comprised in the colloidal particle are ATP and alginate.

31. The composition of claim 19, wherein the ribonucleic acid comprised in the colloidal particle is selected from the group consisting of a messenger RNA, a self-replicating messenger RNA, an interfering RNA, and an antisense RNA.

32. The composition of claim 19, further comprising a biologically active substance other than a ribonucleic acid.

33. The composition of claim 19, wherein the excipient is a salt, an isotonic agent, a serum protein, a buffer, an anti-oxidant, a thickener, an uncharged polymer, a preservative or a cryoprotectant.

34. The composition of claim 19, wherein the ribonucleic acid comprised in the colloidal particle is a messenger RNA.

35. The composition of claim 19, wherein the ribonucleic acid comprised in the colloidal particle is a self-replicating messenger RNA.

36. The composition of claim 19, wherein the ribonucleic acid comprised in the colloidal particle is an interfering RNA.

37. The composition of claim 19, wherein the ribonucleic acid comprised in the colloidal particle is an antisense RNA.

38. A method for transducing a mammalian cell with a ribonucleic acid comprising contacting the cell with a composition according to claim 19.

39. A method for expressing a protein of interest in a mammalian cell, comprising contacting the cell with a composition according to claim 19, wherein the ribonucleic acid contained in the colloidal particle of the composition is a messenger RNA encoding the protein of interest.

40. A method for expressing a protein of interest in a mammalian cell, comprising contacting the cell with a composition according to claim 19, wherein the ribonucleic acid contained in the colloidal particle of the composition is a self-replicating messenger RNA encoding the protein of interest.

41. A method for inhibiting expression of a gene of interest in a mammalian cell, comprising contacting the cell with a composition according to claim 19, wherein the ribonucleic acid contained in the colloidal particle is an interfering RNA directed to a transcript of the gene of interest.

42. A method for inhibiting expression of a gene of interest in a mammalian cell, comprising contacting the cell with a composition according to claim 19, wherein the ribonucleic acid contained in the colloidal particle is an antisense RNA complementary to a transcript of the gene of interest.

43. Process for obtaining colloidal particles according to claim 1 comprising the steps of
(a) preparing an aqueous solution of a chitosan;
(b) preparing an aqueous solution of a ribonucleic acid and a polyanion; and
(c) slowly adding the solution obtained from step (a) to the solution of step (b), such that after addition the particles have negative zeta potential.

44. Process for obtaining colloidal particles according to claim 1 comprising the steps of
(a) preparing an aqueous solution of a chitosan;
(b) preparing an aqueous solution of a polyanion;
(c) preparing an aqueous solution of a ribonucleic acid and, optionally, a polyanion;
(d) slowly adding the solution from step c to the solution of step a, and, optionally, removing excess uncomplexed chitosan subsequent to formation of a dispersion; and
(e) slowly adding the dispersion obtained from step (d) to the polyanion solution, such that after addition the particles have negative zeta potential.

45. Process for obtaining colloidal particles according to claim 1 comprising the steps of
(a) preparing an aqueous dispersion of colloidal particles comprising a chitosan and a polyanion;
(b) preparing an aqueous solution of a ribonucleic acid;
(c) preparing an aqueous solution of a polyanion;
(d) slowly adding the ribonucleic acid solution to the dispersion of step (a) to form a further dispersion; and
(e) slowly adding the dispersion obtained from step (d) to the polyanion solution, such that after addition the particles have negative zeta potential.

46. The process of claim 45, wherein the solution of step b also contains a polyanion.
